(19) United States
(12) Patent Application Publication
(10) Pub. No.: US 2005/0207935 A1
(43) Pub. Date: Sep. 22, 2005

(54) BIO1000 dental handpiece pharmaceutical sterilization lubricant

(76) Inventor: Edwin M. Hayes, Chandler, AZ (US)

Correspondence Address: GUIXIANG YANG, 804 WAVERLY ROAD, KENNETT SQUARE, PA, 19348, US

(21) Appl. No.: 10/805,427

(22) Filed: Mar. 19, 2004

(51) Int. Cl. A61L 2/07

(52) U.S. Cl. 422/26; 433/229

(57) ABSTRACT

About ½ cc or two (2) to three (3) drops of a lubricant (BIO1000) dropped into the rotor drive air feed line of a dental handpiece (FIG. 1) while holding the handpiece upside down protects and lubricates the handpiece bearings and turbine and inhibits rust from forming on the said bearings and turbine, during the auto-clave sterilization process. An additional dosage two (2) to three (3) drops inserted into the rotor drive air feed line of the dental hand piece (FIG. 1) hole after the auto-clave process further lubricates both the bearings and turbine during use. The pre-sterilization dosage and post sterilization dosage can be applied through numerous techniques such as small bottles with needle point like droppers or applicators. The lubricant consists of biodegradable vegetable oil aka BIO 1000. The biodegradability of this lubricant is what makes it different from all other products used for lubrication and sterilization of dental hand pieces.

### REFERENCES CITED

| | | | |
|---|---|---|---|
| 4218216 | August 1980 | Sugai et al. | 433/104. |
| 4920107 | April 1990 | Pera et al. | 422/1. |
| 5131845 | July 1992 | Feldman et al. | 433/104. |
| 5520882 | May 28, 1996 | Brown et al. | |

### BACKGROUND OF THE INVENTION

Field of the Invention

The present invention relates generally to a vegetable oil based lubricant BIO 1000 (aka Marinus) inserted into a dental handpiece prior to and after sterilization to inhibit hand piece corrosion and to lubricate all moving parts.

a. Prior Hand Piece Lubricants and Processes

It is well known that a dental handpiece has to be lubricated periodically to maintain its efficiency and prevent premature failure. The rotor or turbine in the handpiece is supported in bearings, which are subjected to severe wear due to the wide range of speeds and torque loads involved in drilling teeth. Typically, the bearings are lubricated by adding oil in variety of ways, such as from a large oiler connected to the rotor drive air feed line which intermittently drops oil into the inlet air stream where the oil is atomized and then carried to the rotor and bearings entrained in the inlet air.

In a method disclosed in Sugai U.S. Pat. No. 4,218,216 an improved system employed a small oiler inserted in the air feed line having a spring loaded check valve overcome by feed air pressure to allow oil to escape into the feed air, be atomized and continuously lubricate the rotor bearings. However, with the onset of AIDS and the need for increased sterilization, such a system did not function during auto-claving and offered no protection against cross contamination.

By 1991, as disclosed in Feldman U.S. Pat. No. 5,131,845, it was highly recommended that the handpiece be sterilized frequently, and sterilization of the handpiece between patients was becoming a recommended procedure. However, increased sterilization required more frequent lubrication. It was known to lubricate the handpiece both before and after sterilization.

However, the initial lubricants used pre-sterilization and post-sterilization were generally the same products as were previously available prior to the need for increased sterilization. These products did not survive the heat of the auto-clave process itself and were ineffective. Handpiece failures increased and were replaced at an average unit cost of about $150.00.

While Feldman developed an improved method of inserting the oil into the rotor drive air feed line using a squeezable sealed capsule containing the lubricant and his system further prevented the transmission of viral and bacterial matter to the patient and the dentist, it did not advance the art of developing lubricant compositions that could not only withstand the auto-clave cycle, but also, protect the handpiece during the auto-clave cycle by retaining and/or enhancing their boundary lubricating and anti-corrosion characteristics. Feldman merely disclosed that his shell 10 was filled with an extremely light, dental grade, FDA approved, lubricating oil, which was required for the handpiece and widely used in the art. Feldman 7:21-24.

b. Industrial Product Preservatives

It was also known to add ingredients, such as, an anion selected from the group consisting of sulfate, acetate and citrate groups, to aqueous industrial products to inhibit microbiological degradation in such products resulting from the growth of micro-organisms which can cause odors, deterioration and corrosion. Pera U.S. Pat. No. 4,920,107. In an improvement to Nelson U.S. Pat. No. 5,283,005, the biocidal efficacy of such additives were enhanced by mixing a known preservative (pyrithione, used in lubricants) with a known antibiotic (lipopeptide, for treating superficial infections) to inhibit notable bacteria and fungi from forming in the composition itself when used alone or in combination with an inert carrier, such as water, liquid hydrocarbons, ethanol, isopropanol, or the like. 2:63-67. However, there was no disclosure or suggestion of the composition of the present invention, that the disclosed compositions were sterile or could be used as pre-sterilization lubricants for a dental handpiece, or that the boundary lubricating or protective quality of the compositions were enhanced.

### SUMMARY OF INVENTION

Set forth below is a brief summary of the invention in order to achieve the foregoing and other benefits and advantages in accordance with the purpose of the present invention as embodied and broadly described herein.

One aspect of the present invention is a dental handpiece lubricant BIO 1000.

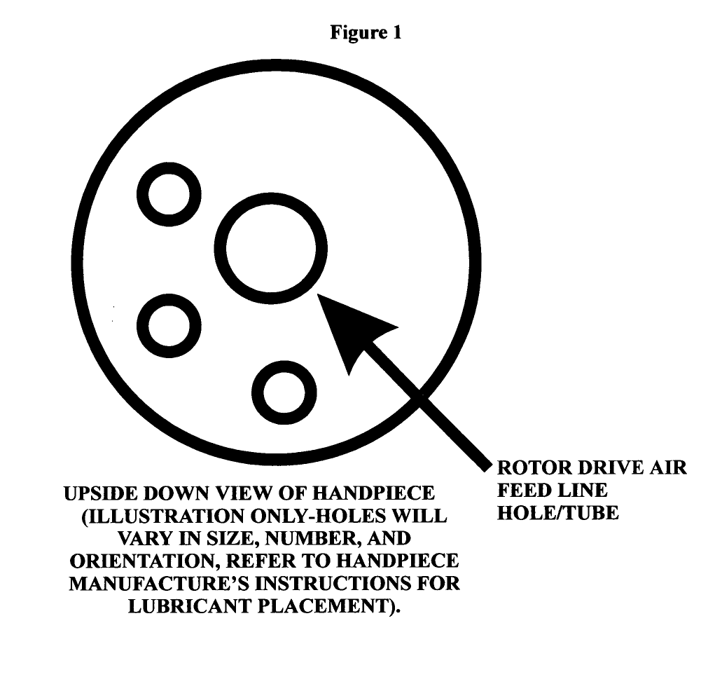

A further aspect of the present invention is the process of sterilizing a dental handpiece having a rotor supported by bearings and a bur hole adapted to receive and hold a bur, by lubricating the handpiece rotor and bearings by inserting either a pre-measured amount or guessed amount of the aforesaid lubrication into the rotor drive air feed line hole/tube (FIG. 1) while holding the handpiece vertically upside down, then, auto-claving the handpiece in either inside or outside a sealed sterilization bag.

A further aspect of the invention is protecting the handpiece in use by inserting either a pre-measured or guessed amount of the aforesaid lubricant into the rotor drive air feed line hole/tube (FIG. 1) following the sterilization process prior to each use or during use.

### DETAILED DESCRIPTION OF THE PREFERRED EMBODIMENT

The composition of the present invention is a specially formulated, vegetable based, lubricant. Characteristics of the Lubricant in this invention are as follows:
1. Color/odor: Green-Blue Tint, Mild petroleum odor.
2. Specific Gravity (H₂O 0.920@15 Deg. C.
3. Vapor Pressure: Not available.
4. Evaporation Rate: Nil@25 Deg. C.
5. Boiling Point:>230 Deg. C.
6. Pour Point (@ 0.01 torr): >−34 Deg. C.
7. Melting Point: Not applicable.
8. Solubility in Water: Insoluble.
9. pH: N/A.
10. Viscosity cSt @ 40° C.: 40.39 to 66.76 @ 100C, 9.4 to 14.76
11. Biodegradability: Based on formulations manufactured, the biodegradability of this product will exceed 90% in 21 days (CEC L-33-A-93).
12. Environmental: This product's low toxicity and ready biodegradability are expected to minimize environmental impact in the event of accidental release.

### WORKING EXAMPLE

The packaging of the invention is conventional. Packaged and sold in small bottles typically ranging from ½ oz. To 2 oz.

Process of Using

The process of using the composition of the present invention is best understood with reference to the drawings.

1. FIG. 1. illustrates the airline connection end of a hand piece. The hand piece is held vertically upside down. The cap of the applicator tip containing the BIO1000 is removed, and the tip of the applicator is inserted into the rotor drive air feed line hole/tube and the bottle is squeezed to allow a dosage of about ½ cc or two (2) to three (3) drops of the lubricant to enter the main air hole of the hand piece.

2. Place the hand piece in a sterilization bag (not shown) and place the bag into a standard auto-clave (not shown).

3. Follow the auto-clave instructions.

4. When the auto-clave cycle is complete, remove the hand piece from the sterilization bag.

5. As seen in FIG. 1 hold the handpiece upside down and reinsert the applicator tip into the rotor drive air feed fine hole/tube and squeeze bottle to allow dosage of two (2) to three (3) drops of the lubricant to enter the air hole/tube (FIG. 1) prior to use of hand piece.

The foregoing description of a preferred embodiment and best mode of the invention is known to applicant at the time of filing the application, and has been presented for the purpose of illustration and description. It is not intended to be exhaustive or to limit the invention to the precision form disclosed, and obviously many modifications and variations are possible in light of the above teaching. The embodiment was chosen and described in order to best explain the principles of the invention and its practical application to thereby enable others skilled in the are to best utilize the invention in various embodiments and with various modifications suited to the particular use contemplated. It is intended that the scope of the invention be defined by the claims appended hereto.

1. The process of sterilizing and lubricating a dental handpiece comprising the steps and fluid of:
(a) Providing a rotor supported in a set of bearings and having a capability of accepting a bur, (a dental hand piece),
(b) Lubricating the rotor and bearing of the handpiece with any amount of BIO1000, (aka trade name Marinus and/or any vegetable based biodegradable lubricants) and
(c) Either inserting the handpiece into a sterilization bag prior to auto-clave or simply applying the BIO1000 to the handpiece and then auto-claving it for sterilization purposes.
(d) Post lubrication of the turbine and bearings using either pre-measured or guessed amounts.

2. The process of claim 1 wherein either a pre-measured amount or any guessed amount.

3. The process of claim 1 wherein the lubricating step further comprises the step of inserting the BIO1000 (modified vegetable oil which has the Trade name of Marinus), hereafter referred to simply as BIO1000, into the rotor drive air feed line hole/tube, (FIG. 1) while holding the handpiece vertically upside down.

4. The process of claim 1 further comprising the step of: after completion of the auto-clave cycle, inserting an amount either pre measured or guessed at into the rotor drive air feed line hole/tube, (FIG. 1).

5. The process of claim 4 wherein the lubricant is BIO 1000.

6. The process of claim 1 further comprising the step of: after completing the auto-clave, cycle, inserting either a pre measured or guessed amount of lubricant (BIO 1000) into the rotor drive air feed line hole/tube (FIG. 1).

7. The process of protecting a dental handpiece having a rotor supported in a bearings and a bur hole constructed and arranged to receive and hold a bur comprising of:
Lubricating the rotor and bearing of the handpiece with either a measured or guessed amount of BIO 1000.
